(19) United States
(12) Patent Application Publication
(10) Pub. No.: US 2024/0002834 A1
(43) Pub. Date: Jan. 4, 2024

(54) ADENINE BASE EDITOR LACKING CYTOSINE EDITING ACTIVITY AND USE THEREOF

(71) Applicants: IUCF-HYU (INDUSTRY-UNIVERSITY COOPERATION FOUNDATION HANYANG UNIVERSITY), Seoul (KR); KOREA UNIVERSITY RESEARCH AND BUSINESS FOUNDATION, Seoul (KR)

(72) Inventors: Sangsu BAE, Seoul (KR); You Kyeong JEONG, BOSTON, MA (US); Seokhoon LEE, Seoul (KR); Jae-Sung WOO, Seoul (KR)

(73) Assignees: IUCF-HYU (INDUSTRY-UNIVERSITY COOPERATION FOUNDATION HANYANG UNIVERSITY), Seoul (KR); KOREA UNIVERSITY RESEARCH AND BUSINESS FOUNDATION, Seoul (KR)

(21) Appl. No.: 18/039,632

(22) PCT Filed: Dec. 1, 2021
(86) PCT No.: PCT/KR2021/017952

§ 371 Date: May 31, 2023

(30) Foreign Application Priority Data
Dec. 1, 2020 (KR) 10-2020-0166028
May 12, 2021 (KR) 10-2021-0061099

(51) Int. Cl. C12N 15/10; C12N 9/78; C12N 15/113; C12N 9/22

(52) U.S. Cl. CPC C12N 15/102; C12N 9/78; C12N 15/113; C12N 9/22; C12Y 305/04004; C12N 2310/20

(57) ABSTRACT

The present invention relates to an adenine base editor from which cytosine editing activity is removed, a method of editing an adenine base, and method of editing an adenine base. In the present invention, it was confirmed that four mutations (V106W, D108Q, F148A and F149A) in adenosine deaminase increase the specificity for adenine editing by reducing cytosine base editing efficiency, and ABE variants were manufactured by introducing respective mutations into a variety of more improved adenine base editors than ABEmax, and tested, confirming that the mutations significantly decrease a cytosine editing effect. Therefore, the adenine base editor according to the present invention is able to more accurately edit adenine, and the method of editing an adenine base can be effectively used in the field of gene therapy that has to accurately edit only adenine in all living organisms including humans, plants and bacteria or novel crop development.

### BACKGROUND

### 1. Field of the Invention

The present invention relates to an adenine base editor from which cytosine editing activity is removed, an adenine base editing composition including the base editor and guide RNA, a method of editing an adenine base and a kit for adenine base editing.

### 2. Discussion of Related Art

Adenine base editors (ABEs) are effective gene editing tools that can convert an A/T pair to a G/C pair without generation of a DNA double-strand break (DSB) or a donor DNA template requirement. They are used when a base is edited not only at the cell level but also at the level of plants or animals, and have been actively verified and developed to be used in gene therapy. The initial practical version of the ABE (ABE7.10) consists of three fused elements. Particularly, the ABE (ABE7.10) consists of a partially inactive Cas nickase (nCas) and a pair of adenosine deaminases (a wild-type tRNA-specific adenosine deaminase (Escherichia coli (wtTadA)-derived TadA), and engineered TadA (eTadA), which is TadA7.10 evolved to work on DNA instead of RNA). However, recently, in ABEs, genome-level single-guide RNA (sgRNA)-dependent off-target DNA editing, transcriptome-level sgRNA-independent off-target RNA editing and an ABE-mediated cytosine deamination effect at an off-target site have been reported. The first off-target effect is caused by incomplete target specificity of a Cas nuclease, and the other two off-target effects are caused by the DNA/RNA-binding characteristics of adenosine deaminase. Therefore, additional engineering of TadA7.10 is required to solve the problem mediated by adenosine deaminase.

To date, several research groups developed a novel version of ABE to which an additional mutation was introduced to reduce an off-target effect on RNA. For example, it was reported that the addition of a F148A mutation in both wtTadA and TadA7.10 reduces random RNA deamination activity. Other groups independently reported that wtTadA in ABE7.10 is mainly responsible for RNA deamination activity, whereas the absence of wtTadA does not affect DNA editing activity. Therefore, it was found that wtTadA was inactivated by introducing an E59A mutation, and an additional mutation (V106W) in TadA7.10 reduces an off-target effect on RNA without reducing DNA targeting activity. Another group completely removes wtTadA, and several mutations were added to TadA7.10 (K20A/R21A or V82G) to reduce an RNA off-target effect (Nat Biotechnol 37, 1041-1048 (2019)). However, as such, many research groups have conducted studies mainly focusing on a decrease in ABE-mediated RNA deamination activity, and in-depth studies on an ABE-mediated cytosine catalytic effect has not been conducted. Accordingly, mutations that can reduce cytosine editing activity and ABE mutants to which these mutations are applied have not been reported.

### SUMMARY OF THE INVENTION

The inventors assumed that, since an engineered eTadA enzyme has a common catalytic site for both adenine and cytosine, active sites and additional mutations occurring nearby can reduce cytosine editing activity without a decrease in adenine editing activity, and main mutations for removing cytosine editing activity were confirmed through rationally designing and testing of dozens of TadA variants. Based on this, the present invention was completed.

Therefore, the present invention is directed to providing cytosine editing activity-removed ABEs.

The present invention is also directed to providing an adenine (A) base editing composition, which includes the ABE; and guide RNA (single guide RNA; sgRNA).

The present invention is also directed to providing a method of editing an adenine (A) base, which includes bringing an adenine base editing composition into contact with a target sequence in vitro.

The present invention is also directed to providing a kit for adenine (A) base editing, which includes an adenine base editing composition.

However, technical problems to be solved in the present invention are not limited to the above-described problems, and other problems which are not described herein will be fully understood by those of ordinary skill in the art from the following descriptions.

To achieve the purpose of the present invention, the present invention provides adenine base editors (ABEs) from which cytosine editing activity is removed, in which an adenosine deaminase variant and a CRISPR associated protein 9 (Cas9) protein are fused, wherein the enzyme variant includes one or more mutations selected from V106W, D108Q, F148A and F149A.

In one embodiment of the present invention, the ABEs may be created by introducing a mutation into any one selected from the group consisting of ABEmax, ABEmax-m, ABE8e, ABE8e-V106W and ABE8.17-m.

In another embodiment of the present invention, the editors may be improved in specificity to adenine base editing.

In still another embodiment of the present invention, the editors may be decreased in RNA editing activity.

The present invention also provides an adenine base editing composition, which includes ABE; and guide RNA (single guide RNA; sgRNA).

In one embodiment of the present invention, the base editing composition may be for substituting adenine (A) with any one selected from the group consisting of guanine (G), cytosine (C) and thymine (T).

The present invention also provides a method of editing an adenine (A) base, which includes bringing the adenine base editing composition into contact with a target sequence in vitro.

The present invention also provides a kit for adenine (A) base editing, which includes the adenine base editing composition.

### BRIEF DESCRIPTION OF THE DRAWINGS

FIG. 1 is a schematic diagram illustrating the editing of target adenine and cytosine, which is mediated by the catalytic activity of an ABE;

FIG. 2 is a graph showing the A4 and C6 base editing efficiency of adenosine deaminase included in various ABEs (left) and ABEs at FANCF and RNF2 sites (right);

FIG. 3 shows the amino acid sequences of adenosine deaminases (TadA) and the result of analyzing the structure thereof, in which FIG. 3A is the result of aligning the sequences of various species-derived wtTadA, TadA7.10 TadA8e, saTadA and 10 TadA orthologs, and FIG. 3B is a structure formed by superimposing an E. coli-derived apo structure (green; PDB code 1Z3A) on the holo structure of saTadA bound to RNA (pink and gray; PDB code 2B3J).

FIG. 4 shows heatmap results (left) obtained by analyzing adenine and cytosine editing induced by 33 ABE variants at FANCF and RNF2 sites and results of analyzing the specificity for adenine editing (right);

FIGS. 5A and 5B show the results of measuring adenine editing efficiency (FIG. 5A) and cytosine editing efficiency (FIG. 5B) of 10 ABE variants to measure adenine and cytosine editing efficiency at a target site in four endogenous FANCF, RNF2, ABLIM3 and CSRNP3 genes after various ABE variants prepared by introducing D108Q or F149A mutations are transfected into HEK293T cells;

FIG. 5C is the graph of comparing the accuracy obtained by dividing the adenine editing efficiency by cytosine editing efficiency in all targets of FIGS. 5A and 5B;

FIG. 6 is the schematic diagram of a cytosine targeting motif-binding region in eTadA;

FIG. 7 shows the average conversion frequency from A to I in four mRNA transcripts after treatment with each of the 10 ABE variants shown in FIG. 5A;

FIG. 8 shows the result of total mRNA sequencing analysis after cells are treated with each of ABE8e, ABE8eW, ABE8eWQ, ABE8s and ABE8sQ among ABE variants of the present invention;

FIG. 9 shows the result of confirming adenine editing rates of ABE8eWQ, ABE8eWA and ABE8eW among the ABE variants of the present invention; and

FIG. 10 shows the result of confirming cytosine editing rates of ABE8eWQ, ABE8eWA and ABE8eW among the ABE variants of the present invention.

### DETAILED DESCRIPTION OF EXEMPLARY EMBODIMENTS

Hereinafter, the present invention will be described in detail.

The inventors confirmed a main mutation reducing cytosine editing activity, and an ABE from which cytosine editing activity is removed by introducing the mutation into adenosine deaminase.

The present invention provides an ABE, which is a cytosine editing activity-removed ABE in which an adenosine deaminase variant and a Cas9 protein are fused, in which the enzyme variant includes one or more mutations selected from the group consisting of V106W, D108Q, F148A and F149A.

The “base editors (BEs)” used herein are single base editing means, and more specifically, refer to adenine base editors (ABEs) or cytosine base editors (CBEs), which are constructed by fusing adenosine deaminase or cytosine deaminase to the N-terminus of Cas9 nickase. The BEs do not induce double-strand breaks, ABEs convert adenine to guanine at a specific site, and CBEs convert cytosine to thymine at a specific site.

The “adenine base editors (ABEs)” are constructed by fusing a natural deaminase (ecTadA) and/or an adenosine deaminase variant (ecTadA*) to the N-terminus of a Cas9 nickase to convert adenine to guanine, and there are ABE6.3, ABE7.8, ABE7.9, ABE 7.10, ABEmax, ABEmax-m, SECURE-ABE, ABE8e, ABE8e-V106W and ABE8.17-m versions according to the type or variation of adenosine deaminase, but the present invention is not limited thereto.

In the present invention, the ABEs mean editors enabling accurate adenine base editing by improving the specificity or accuracy for adenine base editing, and have reduced or removed cytosine base editing activity and RNA off-target activity. The ABEs are preferably obtained by introducing a mutation into any one selected from the group consisting of ABEmax, ABEmax-m, ABE8e, ABE8e-V106W and ABE8.17-m, more preferably, introducing one or more mutations selected from the group consisting of V106W, D108Q, F148A and F149A into the adenosine deaminase of ABEmax, ABEmax-m, ABE8e, ABE8e-V106W or ABE8.17-m, even more preferably, introducing D108Q or F149A, further more preferably, introducing D108Q or F149A into ABE8e-V106W, and most preferably, introducing D108Q into ABE8e-V106W.

The term “adenosine deaminase” used herein refers to an enzyme involved in the removal of an amino group from adenine and generation of hypoxanthine. The enzyme is rarely found in higher animals, but it is reported that small amounts of the enzyme are present in cow muscle, milk and rat blood, and large amounts of the enzyme are present in the intestines of crawfish and insects. The adenosine deaminase may include a natural adenosine deaminase such as ecTadA or an adenosine deaminase variant such as an ecTadA mutant (ecTadA*), and preferably, the variant may include TadA7.10, TadA8e, TadA8s, TadA8.20 or TadA8.17, but the present invention is not limited thereto.

The term “CRISPR associated protein 9 (Cas9) protein” used herein is a protein playing a critical role in immunological defense of specific bacteria against DNA viruses and widely used in genetic engineering applications, and since the main function of the protein is cleavage of DNA, the protein may be applied to modify the genome of a cell. Specifically, CRISPR-Cas9 is simply, rapidly and effectively used to perform editing by recognizing and cleaving a specific base sequence to be used as a third-generation editor, and an operation of inserting a specific gene at a target locus of a genome or stopping the activity of a specific gene. Cas9 protein or gene information may be obtained from a known database such as GenBank of the National Center for Biotechnology Information (NCBI), but the present invention is not limited thereto. In addition, an additional domain may be properly linked to the Cas9 protein according to purpose. In the present invention, the Cas9 protein may not only include wild-typeCas9, but also include all of Cas9 variants as long as they have the function of a nuclease for gene editing.

In addition, in the present invention, the origin of the Cas9 protein is not limited, and as non-limiting examples, the Cas9 protein may be derived from Streptococcus pyogenes, Francisella novicida, Streptococcus thermophilus, Legionella pneumophils, Listeria innocua, or Streptococcus mutans, and is preferably derived from Streptococcus pyogenes.

According to an exemplary embodiment, the inventors confirmed that, in the ABEs according to the present invention, cytosine editing activity is removed and specificity for adenine editing is improved.

More specifically, in one embodiment of the present invention, based on the result confirmed in previous research, as a result of investigating whether a more improved ABE version than ABE7.10 exhibits cytosine deamination activity, it was confirmed that, in all tested ABEs, adenine editing activity as well as cytosine editing is also induced (see Example 2).

In another embodiment of the present invention, to investigate a mutation capable of affecting the elimination of cytosine editing activity through the distinction between adenine and cytosine in adenosine deaminase, based on the sequences of TadA orthologs and three-dimensional structure analysis, variants in which various candidate mutations are introduced into TadA7.10 of ABEmax or ABEmax-m were manufactured, and adenine and cytosine editing efficiency at a target site in HEK293T cells was analyzed. As a result, there were four mutations (that is, V106W, D108Q, F148A and F149A) in which adenine editing efficiency was maintained or slightly decreased and cytosine editing efficiency was substantially reduced, thereby improving the specificity for adenine editing. Further, through an additional test, it was confirmed that D108Q mutation for TadA7.10 enhances the selectivity for adenine editing to the highest level (see Example 3).

According to still another embodiment of the present invention, using TadA8e and TadA8.17 developed to improve adenine editing activity compared to TadA7.10, specifically, ABE8e-D108Q, ABE8e-V106W/D108Q and ABE8.17-D108Q-m (referred to as ABE8eQ, ABE8eWQ, and ABE8sQ) were manufactured by introducing a D108Q mutation into ABE8e, ABE8e-V106W and ABE8.17-m, adenine and cytosine editing efficiency were analyzed by the same method as described above after introduction of F149A into the latest version ABE8e-V106W (referred to as ABE8eWA). As a result, all of the ABE8eQ, ABE8eWQ, ABE8sQ and ABE8eWA exhibited improved adenine editing efficiency and reduced cytosine editing efficiency, and ABE8eWQ and ABE8eWA exhibited very high editing efficiencies and very high specificity. In addition, the effect of reducing RNA off-target editing efficiency for the same ABE variants as above was evaluated, and ABE8eQ, ABE8eWQ and ABE8sQ had a significantly decreased RNA off-target effect. From the above result, it was confirmed that the D108Q mutation effectively reduces RNA deamination activity, and it can be seen that ABE8e-V106W/D108Q most accurately edits adenine (see Example 4).

The above results demonstrate the very sophisticated adenine base editing effect of ABEs according to the present invention and a use thereof.

Therefore, as another aspect of the present invention, the present invention provides an adenine base editing composition, which includes the ABE; and guide RNA (single guide RNA; sgRNA).

The adenine base editing composition of the present invention may substitute adenine (A) with any one base selected from the group consisting of guanine (G), cytosine (C) and thymine (T), and preferably, substitute adenine with guanine.

The “guide RNA (gRNA)” refers to single-stranded RNA serving to find the position of target DNA to be edited and guide a Cas protein to the target DNA, and the guide RNA may be adjacent to a protospacer adjacent motif (PAM), and may include a sequence complementary to the 10 to 25-bp base sequence of DNA to be edited.

In addition, the present invention provides a method of editing an adenine (A) base, which includes bringing the adenine base editing composition into contact with a target sequence in vitro.

In the base editing method according to the present invention, the target sequence may include a target base to be edited, and the target base to be edited may be another base excluding adenine, and preferably guanine, which may be point-mutated to adenine, but the present invention is not limited thereto.

In addition, the present invention provides a kit for adenine (A) base editing, which includes the adenine base editing composition.

In the present invention, the kit may include materials (reagents) required for base editing such as a buffer and deoxyribonucleoside-5-triphosphate (dNTP) in addition to the base editing composition. In addition, the optimal amount of a reagent used in a specific reaction of the kit may be easily determined by one of ordinary skill in the art acquiring the disclosure in the specification.

Hereinafter, to help in understanding the present invention, exemplary examples will be suggested. However, the following examples are merely provided to more easily understand the present invention, and not to limit the present invention.

### EXAMPLES

### Example 1. Experimental Materials and Experimental Methods

1-1. Construction of Plasmid for Expressing ABE Variant

The inventors constructed an ABE variant-expressing plasmid based on pCMV_ABEmax_P2A_GFP (Addgene #112101), pCMV-ABEmax (TadA E59A) (Addgene #125648), pCMV-ABEmax (TadA, eTadAE59A) (Addgene #125662), pCMV-ABEmax (TadA E59A, eTadAR47Q) (Addgene #125657), pCMV-ABEmax (TadA E59A, eTadAD108Q) (Addgene #125655), pCMV-ABEmaxAW (Addgene #125647), ABE8e (Addgene #138489), ABE8e (TadA-8e V106W) (Addgene #138495), or ABE8.17-m (Addgene #136298). To obtain a vector for ABE expression, 1 μg of pCMV_ABEmax_P2A_GFP, 1 unit of NotI-HF (New England Biolabs, catalog number: R3189L) and 1 unit of BglII (Enzynomics, catalog number: R010S) were mixed to a final volume of 50 μl. Subsequently, the mixture was incubated at 37° C. for 1 hour to induce a cleavage reaction, and then subjected to electrophoresis to separate a cleaved product by size, followed by purifying the product using a gel extraction kit, Expin™ Gel SV mini (GeneAll, catalog number: 102-102). The purified result was mixed with 2U of T5 exonuclease (New England Biolabs, catalog number: M0363S), 12.5U of Phusion DNA polymerase (Thermo Fisher Scientific, catalog number: F530L), 2kU of Taq DNA ligase (New England Biolabs, catalog number: M0208S), 0.2M Tris-HCl (pH 7.5), 0.2M MgCl₂, 2 mM dNTPs, 0.2M dithiothreitol, 25% PEG-8000 and 1 mM NAD to a final volume of 10 and a synthesized PCR product including the purified product and a variant to be detected was added and mixed, and incubated at 50° C. for 1 hour. Subsequently, the product was transformed into 100 μl of DH5a competent cells. The transformed single colonies were inoculated into an LB medium containing an antibiotic, and plasmids were isolated from the cells using a DNA prep kit (Enzynomics, EP101-200N).

1-2. Transfection Using Liposome

HEK293T (ATCC CRL-3216™) cells were incubated in a DMEM medium supplemented with 10% FBS and 1% ampicillin at 37° C. under a 5% CO₂ condition. A cell density was estimated by hemocytometer and microscopic observation. Before transfection, HEK293T cells were dispensed into a 24-well plate at a density of 1×10⁵ cells per well and incubated for 24 hours, and a mixture of 2 μL of the Lipofectamine 2000 reagent (Thermo Fisher Scientific, 11668019), 1 μL of plasmid DNA (750 ng of ABE expression plasmid and 250 ng of sgRNA expression plasmid) and a serum-free medium was added to the cells. Genomic DNA was isolated 72 hours after transfection.

1-3. Transfection by Electroporation

The ABEmax expression plasmid (500 ng) and the sgRNA expression plasmid (170 ng) were introduced into 2×10⁵ cells by electroporation using a Neon′ Transfection System 10 μL kit (Thermo Fisher Scientific, MPK1025). Appropriate electroporation parameters (1,500V-20 ms-2pulses for HEK293T cells) were used according to the manufacturer's protocol. In addition, genomic DNA was isolated 72 hours after transfection.

1-4. Sequence Alignment of TadA Orthologs

Protein BLAST at the National Center for Biotechnology Information (NCBI) was used to search for TadA orthologs. The E. coli wtTadA sequence (GenBank ID: WP_001297409.1) was used as an input sequence, and 10 orthologs showing ˜40% sequence identities were selected. The accession numbers and species for the selected sequences are as follows: vsTadA, WP_127165941.1, Veillonella sp. CHU732; ssTadA, WP_105128341.1, Streptococcus suis; asTadA, WP_067866801.1, Acinetobacter sp. SFB; bfTadA, WP_073388705.1, Butyrivibrio fibrisolvens; sxTadA, WP_107541930.1, Staphylococcus xylosus; cbTadA, WP_111988333.1, Cellvibrionaceae bacterium AOL6; dfTadA, WP_117494436.1, Dorea formicigenerans; ocTadA, WP_047980683.1, Ornithinibacillus contaminans; osTadA, WP_077602817.1, Oceanobacillus sojae; pgTadA, WP_026908502.1, Paucisalibacillus globulus.

1-5. Targeted Deep Sequencing

HEK293T cells were centrifuged, and a cell pellet was resuspended in 100 μl of Proteinase K extraction buffer [40 mM Tris-HCl (pH 8.0), 1% Tween-20, 0.2 μM EDTA, 10 mg Proteinase K, 0.2% Nonidet P-40], and incubated at 60° C. for 15 minutes, followed by incubation at 98° C. for 5 minutes. Genomic DNA isolated from the HEK293T cells was amplified using KOD-Multi & Epi (TOYOBO, KME-101), and the resulting PCR product was analyzed using an Illumina MiSeq instrument. The MiSeq results were analyzed using BE-Analyzer (http://www.rgenome.net/be-analyzer/).

1-6. Targeted RNA Sequencing

In a 24-well plate, HEK293T cells were transfected with 500 ng of the ABE expression plasmid and 170 ng of sgRNA through electroporation, and after 24 hours, the cells were washed with DPBS. RNA was isolated by using a NucleoSpin® RNA Plus kit (MACHEREY-NAGEL, 740984. 250) according to the manufacturer's instructions, and cDNA synthesis was performed through reverse transcription using ReverTra Ace-α-™ (TOYOBO, FSK-101) according to the manufacturer's instructions. For cDNA amplification, PCR was performed with KOD-Multi & Epi (TOYOBO, KME-101), and the PCR product was analyzed using an Illumina MiSeq instrument. To obtain the percentage of editing adenosine to inosine, the number of adenosines converted to guanosines was divided by the total number of adenosines in the products.

### Example 2. Measurement of Cytosine Deamination Activity in Various ABE Variants

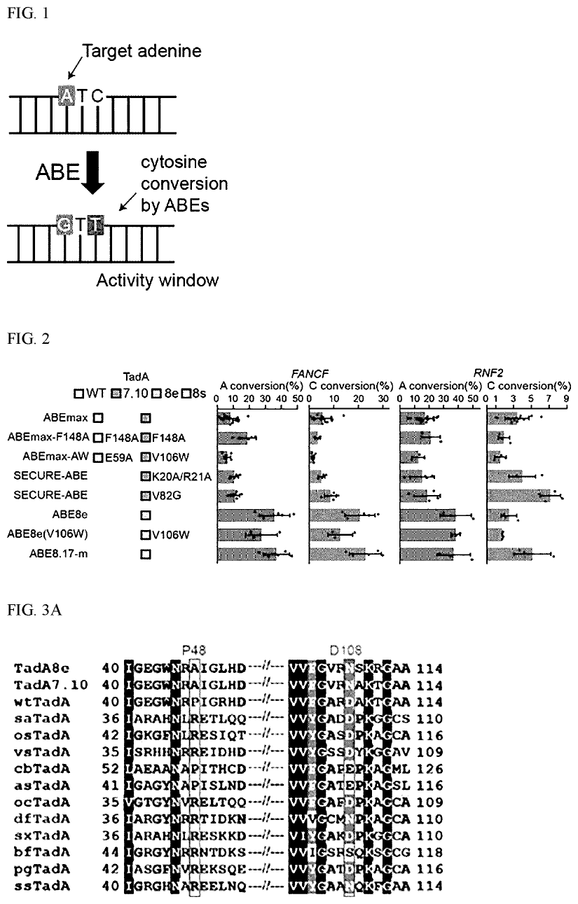

In previous research, the inventors confirmed that the adenine base editor (ABE), ABE7.10, can not only edit adenine, which is the original target base, in a narrow window (5^{th} to 7^{th} position from the 5′ terminus of the Cas9 target range) of a preferred motif (TC*N) as shown in FIG. 1, but also when there is a TC target motif, cytosine deamination was catalyzed to substitute cytosine. In addition, it was confirmed that cytosine base substitution by such cytosine deaminase is shown not only in ABE7.10, but also in previous versions thereof (ABE6.3, ABE7.8 and ABE7.9) and the more optimized version (ABEmax).

Therefore, recently, the inventors attempted to investigate whether cytosine deamination activity is also exhibited in ABE variants developed by variously modifying the adenosine deaminase TadA. Types of the ABE variants used in the experiment are as follows, and the detailed information on TadA included in each variant is shown in FIG. 2: 1) versions (i.e., ABEmax-F148A, ABEmax-AW, and SECURE-ABEs) developed to reduce an ABE-mediated RNA off-targeting effect, 2) versions (i.e., ABE8e and ABE8e-V106W) including the TadA8e variant exhibiting increased deamination activity, and 3) a version including TadA8s exhibiting improved editing activity (i.e., ABE8.17-m).

To this end, all ABE variants were tested against two representative endogenous targets (FANCF and RNF2) having both an adenine residue and a cytosine target motif within the editing window in human HEK293T cells, and subjected to high-throughput sequencing analysis, thereby obtaining the following results.

First, as shown in FIG. 2, all of the tested ABE variants were shown to still induce cytosine editing in addition to adenine editing, but it was confirmed that, in the case of ABEmax-F148A and ABEmax-AW, cytosine editing efficiencies were slightly reduced. It was determined that this supports the hypothesis of the inventors in that further engineering of eTadA can eliminate or minimize cytosine deamination activity.

Second, although all ABE8 variants showed adenine editing efficiency which is greatly increased compared with the previous versions, cytosine editing efficiency was also increased. Nevertheless, it was confirmed that ABE8e-V106W shows decreased cytosine editing efficiency compared with ABE8e.

Third, it was seen that deactivation or elimination of wtTadA does not hinder DNA editing activity, and rather, a wtTadA-free ABEmax version (ABEmax-m) showed higher DNA editing activity than the original ABEmax.

### Example 3. Confirmation of Mutations in TadA Having Significant Effect on Cytosine Editing Efficiency

The inventors thought that some of TadA orthologs may have already evolved to circumvent cytosine editing and tried to confirm main mutations promoting the distinction between adenine and cytosine in the adenosine deaminase TadA. Therefore, to this end, the amino acid sequences of TadA orthologs derived from various species were investigated.

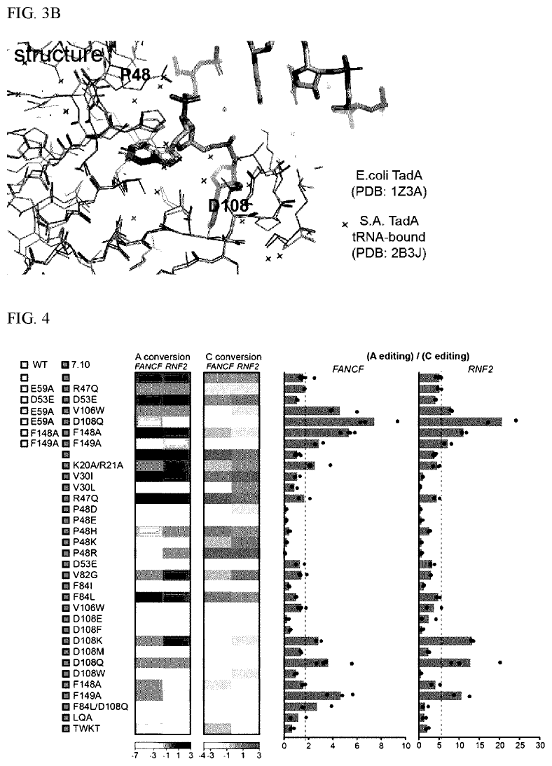

As a result, as shown in FIG. 3A, based on the aligned amino acid sequences of the TadA orthologs and the tertiary structure of Staphylococcus aureus TadA (saTadA) binding to a tRNA fragment of FIG. 3B, it was found that several residues substantially inside and outside active sites are variously mutated between orthologs. For example, it can be seen that P48 of E. coli wtTadA was substituted with arginine in most of the TadA orthologs, and D108 was substituted with asparagine, glutamate or serine in other orthologs. In addition, the saTadA structure of FIG. 3B provided insight into the conformational change in an RNA substrate required for deamination of cytosine smaller than adenine. For adenine deamination activity, the hexagonal ring of adenine needs to be located deep inside the adenine-binding pocket, similar to what is shown in the saTadA structure with a purine base bound to the pocket. However, for cytosine deamination, the pyrimidine ring needs to be located at the same position as the hexagonal ring of a purine base in the structure, resulting in a shift of the sugar-phosphate backbone toward the edge of the pocket. Therefore, the inventors decided to substitute P48 and D108 with larger residues capable of preventing the DNA backbone from approaching the pocket edge. In addition, V30 and F84 located in the adenine-binding pocket were substituted with isoleucine and leucine found at the corresponding locations in many TadA orthologs. In addition, mutations that have been previously tested and shown to incompletely reduce RNA editing activity, such as an R47Q mutation, which maintains DNA on-target editing activity, and a D53E mutation which reduces RNA editing activity in vitro, were introduced.

Subsequently, each candidate mutation which may affect a cytosine editing effect of an ABE was introduced into TadA7.10 of ABEmax or ABEmax-m, and then the nucleotide conversion activity of each ABE variant was tested at target sites in FANCF and RNF2 genes by transfection into HEK293T cells.

As a result, as shown in FIG. 4, it was confirmed that when the adenine and cytosine editing efficiencies of the ABE variants were normalized to that of ABEmax, adenine and cytosine editing efficiencies were simultaneously increased or decreased in most of the mutations. However, from the right graph showing the accuracy value obtained by dividing adenine editing efficiency by cytosine editing efficiency, it was found that, in four mutations (that is, V106W, D108Q, F148A and F149A), the cytosine editing efficiency is substantially decreased, and the adenine editing efficiency is maintained or slightly reduced, resulting in higher specificity for adenine editing. In addition, the inventors tested the activity of ABE variants including the four mutations found as above against the target sites of two additional endogenous genes, and identified that ABEmax-m including TadA7.10-D108Q (hereinafter, ABEmaxQ-m) shows the highest selectivity for adenine editing.

In summary, from the above results, a TadA7.10 variant, such as TadA7.10-D108Q, showing improved selectivity for adenine or cytosine editing, was found.

### Example 4. Selection of Optimized Adenine Base Editor from which Cytosine Editing Activity is Removed

In Example 3, ABEmaxQ-m showed a highly decreased cytosine editing effect, but also showed a decreased adenine editing efficiency compared to ABEmax. The inventors tested TadA8e and TadA8.17 developed to improve editing activity instead of TadA7.10 in order to compensate for the lower editing efficiency for adenine. Specifically, ABE8e-D108Q, ABE8e-V106W/D108Q and ABE8.17-D108Q-m were prepared by introducing the D108Q mutation confirmed in Example 3 into ABE8e, ABE8e-V106W and ABE8.17-m, and named ABE8eQ, ABE8eWQ and ABE8sQ, respectively. In addition, the latest version, ABE8e-V106W/F149A was prepared by introducing a F149A mutation into ABE8e-V106W, and named ABE8eWA. A total of four endogenous target sites (FANCF, RNF2, ABLIM3 and CSRNP3) were tested using all of the prepared ABE variants, and then subjected to high-throughput sequencing analysis.

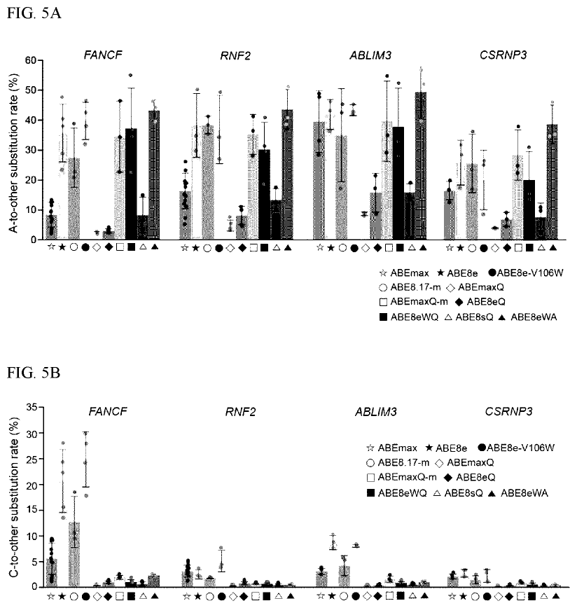

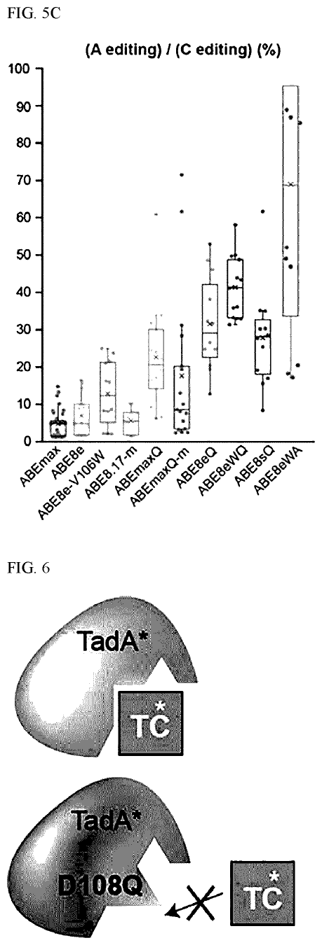

As a result, as shown in FIGS. 5A and 5B, all of the ABE8eQ, ABE8eWQ, ABE8sQ and ABE8eWA variants exhibited improved adenine editing efficiency and decreased cytosine editing efficiency. Particularly, as shown in FIG. 5C, which is a graph of comparing accuracy values obtained by dividing adenine editing efficiency by cytosine editing efficiency for all ABE variants, ABE8eWQ and ABE8eWA exhibit very high editing efficiency and specificity. As shown in FIG. 6, this result shows the V106W and D108Q mutations in TadA8e of the variants exhibited synergistic effects, and thus the D108Q mutation played a critical role in hindering TadA protein binding to a TC motif.

Next, the ABE-mediated RNA off-target editing efficiency of all ABE variants was evaluated. To this end, each ABE variant was transfected into HEK293T cells, and the A to I conversion frequency was measured in four representative RNA transcripts (CCNB1IP1, AARS1, PERP and TOPRS), and then high-throughput sequencing was performed.

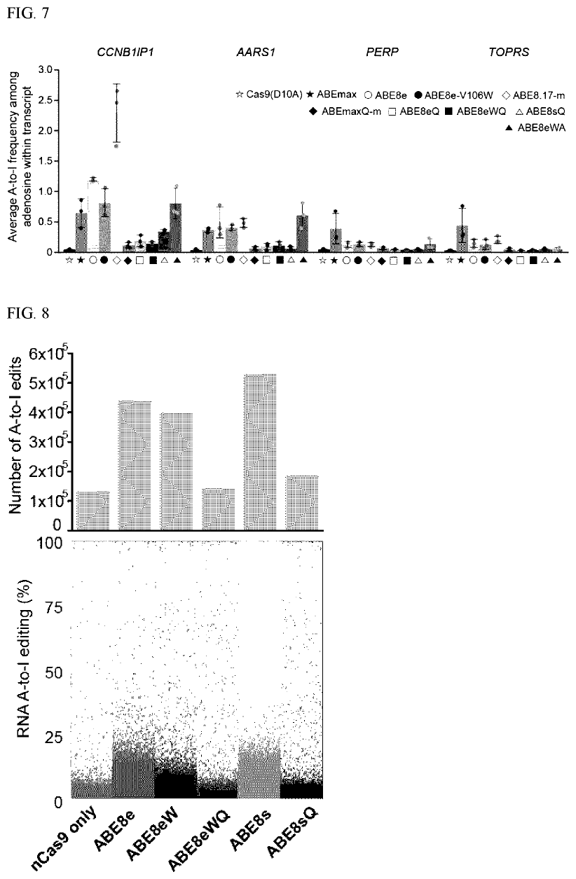

Consequently, as shown in FIG. 7, the previously developed ABE8e and ABE8.17-m versions showed a higher RNA off-target effect than ABEmax, and as previously reported, it was confirmed that ABE8eW exhibits a reduced RNA off-target effect compared to ABE8e. In addition, in the case of ABE8eWA, the RNA off-target effect was similar to that of ABE8e.

ABE8eQ, ABE8eWQ and ABE8sQ had a significantly decreased RNA off-target effect, which indicates that the D108Q mutation effectively reduces RNA deamination activity. D108Q can reduce the binding affinity of TadA8e to RNA, but not to DNA, which is because a carboxyl group of D108 forms a hydrogen bond with the 2′ hydroxyl group of the bound RNA in the saTadA-RNA structure.

Taken together, the above results show that D108Q is a very important mutation that reduces both cytosine editing and RNA deamination editing efficiencies, and ABE8eQ, ABE8eWQ and ABE8sQ are the optimized ABE versions, among which ABE8eWQ is the most effective and optimal version.

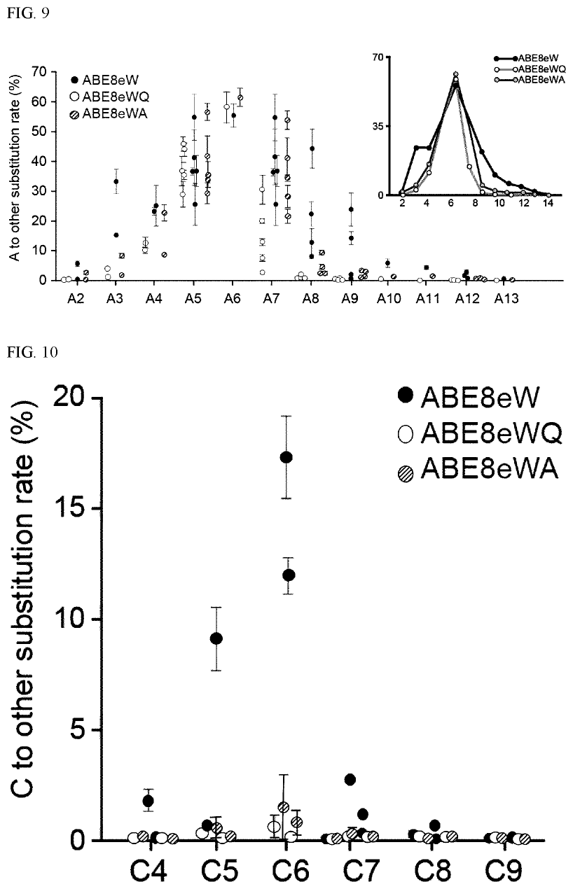

In order to support the above conclusion, cells were treated with a control (nCas-treated group) and ABE8e, ABE8eW, ABE8eWQ, ABE8s and ABE8sQ among the ABE variants of the present invention, and then subjected to total mRNA sequencing, and as a result, it was confirmed that, as shown in FIG. 8, ABE8eWQ shows an RNA deamination effect at a level most similar to the control. As a result of confirming the adenine (FIG. 9) or cytosine (FIG. 10) editing effect according to the positions of ABE8eWQ, ABE8eWA and ABE8eW among the variants of the present invention, it was confirmed that ABE8eWQ and ABE8eWA did not exhibit a cytosine editing effect, but had a similar adenine editing effect to that of ABE8eW.

According to the experimental results as above, the inventors confirmed that, while ABE8eWA was manufactured based on the latest version ABE8e-V106W in the same manner as ABE8eWQ, it has the highest adenine editing efficiency, but little cytosine editing efficiency, and still has an RNA off-target effect, compared to ABE8eWQ, thus, determining the most accurate ABE is ABE8eWQ.

In the present invention, four mutations (V106W, D108Q, F148A and F149A) in adenosine deaminase, which increase specificity for adenine editing by reducing cytosine base editing efficiency, were confirmed, and an ABE variant prepared by introducing each mutation to a more improved version of various ABEs than ABEmax was manufactured and tested, thereby confirming a significant decrease in cytosine editing effect by the mutations. Therefore, since the ABEs according to the present invention enable more accurate adenine editing, an adenine base editing composition including the ABE and sgRNA can be effectively used in the field of gene therapy or new crop development in which only adenine should be accurately edited in all living things, including humans, plants and bacteria.

What is claimed is:

1. A cytosine editing activity-removed adenine base editor in which adenosine deaminase variant and a CRISPR-associated protein 9 (Cas9) protein are fused,
wherein the variant has one or more mutations selected from the group consisting of V106W, D108Q, F148A and F149A.

2. The adenine base editor of claim 1, wherein the mutation is introduced into any one selected from the group consisting of ABEmax, ABEmax-m, ABE8e, ABE8e-V106W and ABE8.17-m.

3. The adenine base editor of claim 1, which has improved specificity for adenine base editing.

4. The adenine base editor of claim 1, which has reduced RNA editing activity.

5. An adenine (A) base editing composition, comprising:
the adenine base editor of claim 1; and
guide RNA (single guide RNA; sgRNA).

6. The composition of claim 5, which substitutes adenine (A) with any one base selected from the group consisting of guanine (G), cytosine (C) and thymine (T).

7. A method of editing an adenine (A) base, comprising:
bringing the adenine base editing composition of claim 5 into contact with a target sequence in vitro.

8. A kit for adenine (A) base editing, comprising the adenine base editing composition of claim 5.
